(19) United States
(12) Patent Application Publication
(10) Pub. No.: US 2026/0215950 A1
(43) Pub. Date: Jul. 30, 2026

(54) NASAL STENT

(71) Applicant: PS Devices, LLC, Lyndhurst, OH (US)

(72) Inventor: Bahman Guyuron, Lyndhurst, OH (US)

(21) Appl. No.: 19/142,306

(22) PCT Filed: Dec. 11, 2023
(86) PCT No.: PCT/US2023/083310

§ 371 Date: Jun. 23, 2025

Related U.S. Application Data
(60) Provisional application No. 63/435,023, filed on Dec. 23, 2022.

(51) Int. Cl. A61F 5/058; A61F 5/08

(52) U.S. Cl. CPC A61F 5/05891; A61F 5/08

(57) ABSTRACT

An intranasal device including a nostril dilator and a nasal splint joined by a flexible connector. The nostril dilator may be anatomically shaped. The flexible connector facilitates correct positioning of the nasal splint and makes the intranasal device adaptable to a wide range of human anatomical variations. A flange designed to rest on the front of a nostril may be disposed on an anterior end of the nostril dilator. The intranasal device may have two arms each of which includes a nostril dilator and a nasal splint joined by a flexible connector. The joining structure may be a band and may be bowed so as not to rest on the columella. The band may also facilitate gripping or hooking onto the intranasal device for the purpose of pulling it out. The intranasal device may be used without stitches.

### BACKGROUND

Nasal splints are used following surgery to hold the septum in place over a healing period of days or weeks. These nasal splints are usually held in place with sutures. The patient returns to the surgeon after the healing period to remove the nasal splints.

### BRIEF DESCRIPTION OF THE DRAWINGS

Aspects of the present disclosure are best understood from the following detailed description when read with the accompanying figures.

FIG. 1 is a side view of an intranasal device according to some embodiments of the present disclosure.

FIG. 2 is a top view of the intranasal device of FIG. 1.

FIG. 3 is a perspective view of the intranasal device of FIG. 1.

FIG. 4 is a front view of the intranasal device of FIG. 1.

FIG. 5 is a flow chart of a method according to some embodiments of the present disclosure.

### DETAILED DESCRIPTION

The present disclosure provides embodiments, or examples, for implementing different features of this disclosure. Specific embodiments or examples and are not intended to be limiting.

The present disclosure provides an intranasal device that may be inserted after nasal surgery to improve healing. The device maintains structure and eliminates dead space in which blood might otherwise accumulate, convert to scar tissue, and potentially block the airway. In some embodiments, the intranasal device includes a nostril dilator and a nasal splint joined by a flexible connector. The nasal splint comprises a septum support and may have an additional structure that maintains a breathing passage and braces the septum support. In some embodiments, the nostril dilator is anatomically shaped. In some embodiments, the shape of the nostril dilator prevents or ameliorates the pooling of blood in the nasal vestibule. In some embodiments, the nostril dilator comprises a bulge between its anterior end and its posterior end. The bulge may resist extraction of the intranasal device. In some embodiments, the nostril dilator has a larger outer diameter than the breathing passage maintained by the nasal splint.

In some embodiments, the nostril dilator, the nasal splint, and the flexible connector are all formed of a single biocompatible material. By virtue of its connection to the nostril dilator and its flexibility, the flexible connector facilitates correct positioning of the nasal splint and makes the intranasal device adaptable to a wide range of human anatomical variations. In some embodiments, the flexible connector is a ribbon. In some embodiments, the flexible connector has a length and allows the septum support to be displaced laterally with respect the nostril dilator.

In some embodiments, a flange is disposed on an anterior end of the nostril dilator. The flange is shaped to rest on the front of a nostril and to limit a depth to which the intranasal device may be inserted into the nostril. The flange helps secure the intranasal device without the use of stitches. In some embodiments, the flange extends upward in the direction of the tip of the nose.

In some embodiments, the intranasal device has two arms each of which includes a nostril dilator and a nasal splint. A joining structure connects the two arms. In some embodiments, the joining structure is a flexible connector. In some embodiments, the joining structure is a band. In some embodiments, the joining structure is bowed so as not to rest on the columella. Preventing the joining structure from resting against the columella facilitates healing. The forward bow may also facilitate gripping or hooking onto the intranasal device for the purpose of pulling it out. If the nostril dilators have flanges on their anterior ends, the joining structure may bow forward of the flanges.

The nostril dilators are in a flexible relationship with the nasal splints. The nostril dilators improve the shape of the nostrils and preventing collapse of the nostrils during healing. The nasal splints have a soft flap that prevents scar tissue from forming between the septum and the turbinates. The soft nature of these nasal splints offers easy extraction without significant patient discomfort. Prior art nasal splints are challenging to remove, requiring special tools and medical professionals. Those prior art nasal splints are usually retained with sutures that often cause patients severe discomfort. The intranasal devices of the present disclosure avoid this discomfort and can be removed without any tools by untrained individuals such as ordinary patients and their family members.

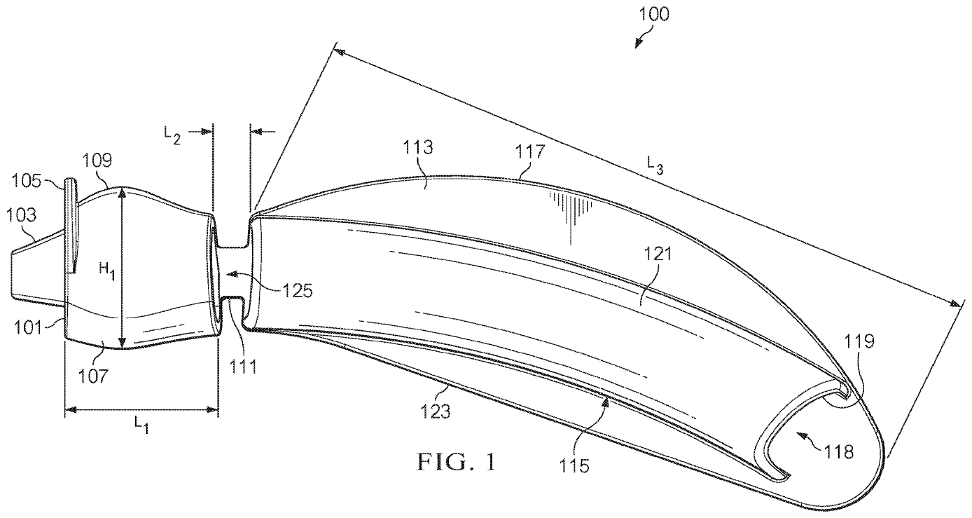

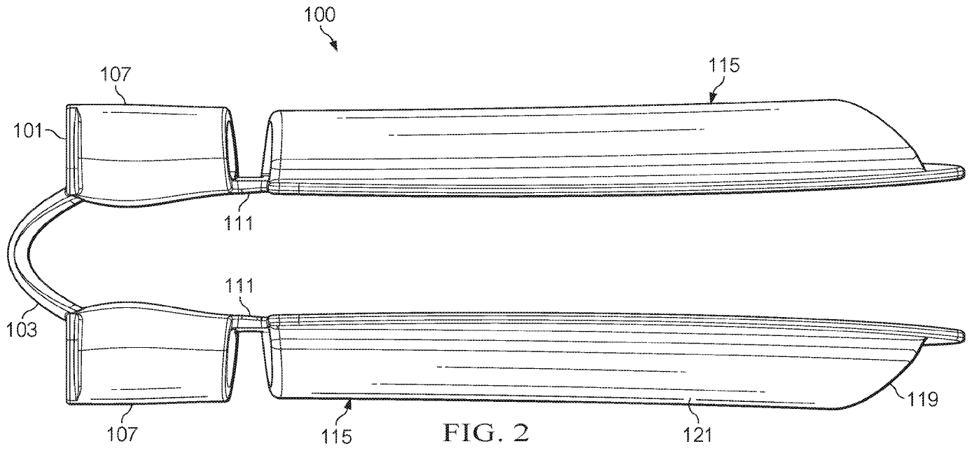

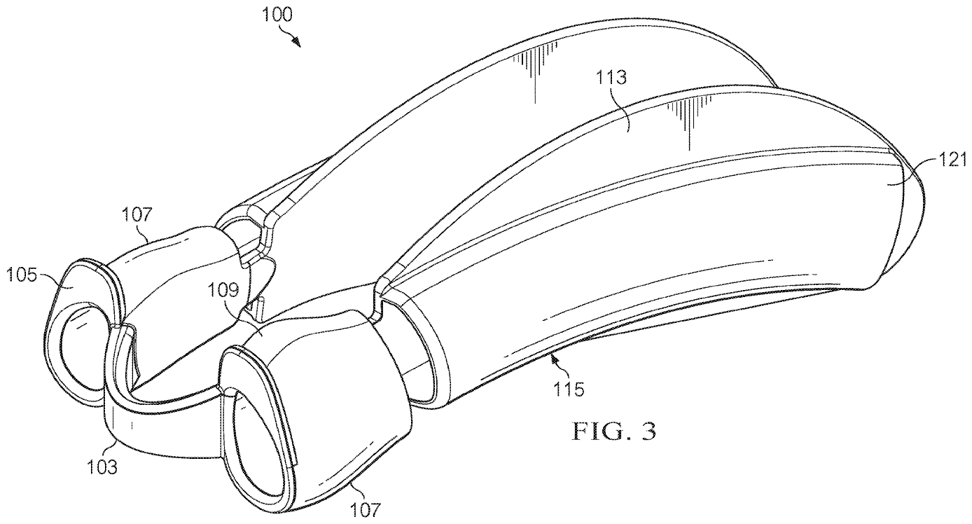

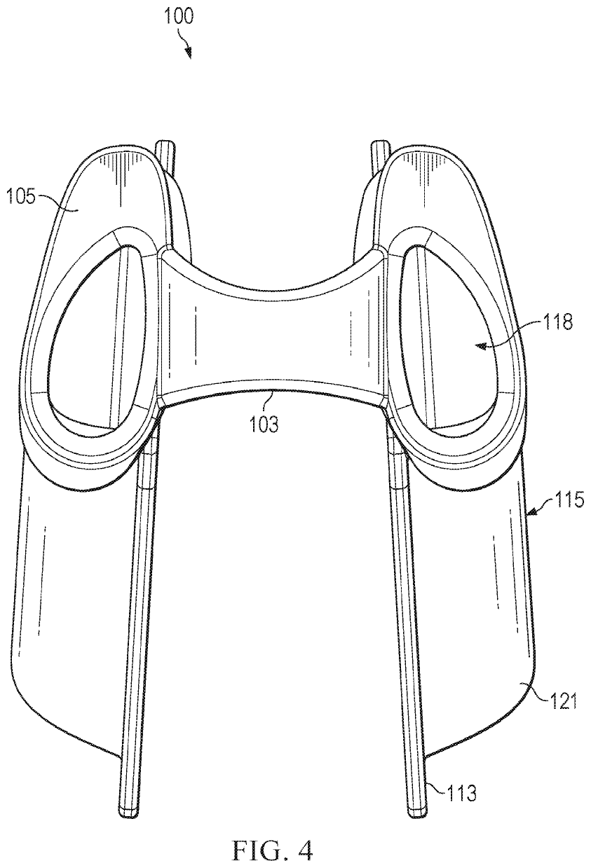

FIGS. 1-4 illustrate an intranasal device 100 according to some embodiments of the present disclosure. FIG. 1 provides a side view, FIG. 2 provides a top view, FIG. 3 provides a perspective view, and FIG. 4 provides a front view. The intranasal device 100 includes a nostril dilator 107 that is connected to a nasal splint 115 by a flexible connector 111.

The nostril dilator 107 has a bulge 109 between its anterior end 101 and its posterior end 125. A flange 105 is attached to the anterior end 101 and extends upward. The nostril dilator 107 has a shape corresponding to human nasal anatomy. With reference to FIG. 4, it can be seen that the nostril dilators 107 of the left and right sides are asymmetrical and are mirror images of one another. Lower portions of the nostril dilators 107 are wider than upper portions. Outer sides of the nostril dilators 107 are more bowed than their inner sides.

The nostril dilators 107 have a maximum height HI that occurs at the bulge 109. In some embodiments, the height HI is in the range from about 1.0 cm to about 2.5 cm. In some embodiments, the height HI is in the range from about 1.5 cm to about 2.0 cm, e.g., about 1.7 cm. In some embodiments, the nostril dilators 107 have a length Li in the range from about 1.0 cm to about 2.0 cm. In some embodiments, the length Li is in the range from about 1.2 cm to about 1.6 cm, e.g., about 1.4 cm.

The nostril dilators 107 of the left and right sides are connected by a band 103.

The band 103 bows forward of the nostril dilators 107 and the flanges 105. The band 103 may connect to the inner sides of the nostril dilators 107. In some embodiments, the band 103 has a length (when extended flat) that is in the range from about 1.0 cm to about 3.0 cm. In some embodiments, the band 103 has a length 1.5 cm that is in the range from about 1.2 cm to about 2.0 cm, e.g., about 1.5 cm. The band 103 is flexible. With reference to FIG. 1, the flexibility allows the nasal splint 115 to be displaced laterally (into or out of the page) relative to the nostril dilator 107. The amount of lateral displacement may be equal to the length of the band 103 or even greater than the length of the band 103 provided that the band 103 is elastic.

The nasal splint 115 comprises a platelike member 113 and a partial tube structure 121. The partial tube structure 121 maintains a breathing passage 118 and is operative to bias the platelike member 113 against a septum to support the septum. The platelike member 113 is shaped to provide a septum splint. That shape may include a relatively flat lower edge 123 and a curved upper edge 117. The partial tube structure 121 may extend outward from the platelike member 113 and curl downward to maintain a breathing passage. In some embodiments, the partial tube structure 121 is a semi-tube. In some embodiments, the partial tube structure 121 has a tapered end 119 that facilitates insertion.

The partial tube structure 121 may cooperate with the platelike member 113 to fully enclose the breathing passage 118. The nasal splint 115 may be of the type know in the art as a Doyle splint. In some embodiments, the nasal splint 115 has a length L₃ in the range from about 5 cm to about 8 cm. In some embodiments, the length L₃ is in the range from about 6.0 cm to about 7.0 cm, e.g., about 6.4 cm.

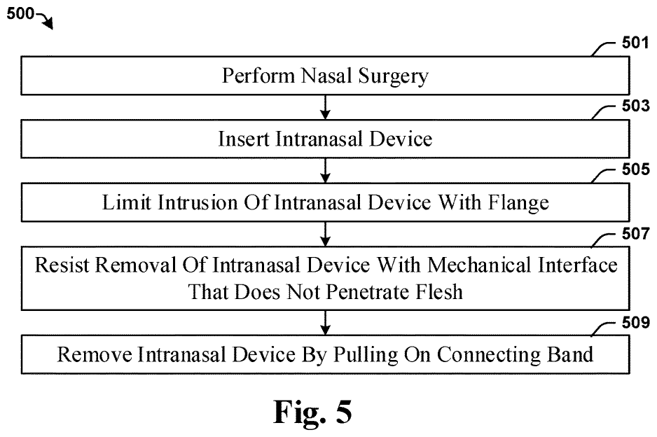

FIG. 5 provides a flow chart of a method 500 in which the nasal splint 115 or the like is used. The method 500 begins with act 501, performing a nasal surgery. The nasal surgery may be or comprise, for example, rhinoplasty, turbinate reduction, septoplasty, or rhinoseptoplasty.

The method 500 continues with act 503, inserting an intranasal device. In some embodiments, this comprises inserting two arms of the intranasal device 100 into two respective nasal passages. The nasal splints 115 are placed against the sides of the septum. The nostril dilators 107 are placed in the nasal vestibules. In some embodiments, the intranasal device 100 is insert to a point where the flanges 105 rest on the fronts of the nostrils.

Act 505 and act 507 both relate to maintaining a position of the intranasal device 100 while healing is underway. Act 505 is using the flanges 105 to keep the arms from intruding too deeply into the nostrils. Act 507 is using non-intrusive, non-flesh penetrating mechanical means to keep the arms of the intranasal device 100 from inadvertently coming out of the nostrils. The term “non-flesh penetrating” is used to distinguish stitches which are not needed when using the intranasal device 100. The mechanical means may include interfacing between the nostril dilators 107 and the nasal vestibules.

Act 509 is removing the intranasal device 100 by pulling on the connecting band 103. In some embodiments, this comprises grasping the connecting band 103 and pulling it out. In some embodiments, this comprises inserting an object between the connecting band 103 and the columella and using that object to pull out the intranasal device 100. The object may be, for example, a pen or pencil.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. An intranasal device, comprising:
a nostril dilator;
a nasal splint comprising a septum support and partial tube structure configured to maintain a breathing passage; and
a flexible connector between the nostril dilator to the nasal splint.

2. The intranasal device of claim 1, wherein the nostril dilator has a larger outer diameter than the breathing passage.

3. The intranasal device of claim 1, wherein the flexible connector has a length and allows the septum support to be displaced laterally with respect the nostril dilator.

4. The intranasal device of claim 1, wherein the flexible connector is a ribbon.

5. The intranasal device of claim 1, wherein the nostril dilator, the nasal splint, and the flexible connector are formed of a biocompatible material.

6. The intranasal device of claim 1, further comprising a flange on an anterior end of the nostril dilator.

7. The intranasal device of claim 6, wherein the flange extends upward and is shaped to rest in front of a nostril within which the nostril dilator is disposed.

8. The intranasal device of claim 6, wherein the nostril dilator comprises a bulge between the anterior end and a posterior end.

9. The intranasal device of claim 1, wherein the nostril dilator has a shape adapted to prevent blood from pooling in a human nasal vestibule.

10. A bilateral intranasal device, comprising two intranasal devices according to claim 1 joined together.

11. The bilateral intranasal device of claim 10, wherein:
one of the intranasal devices has a flange on an anterior end; and
the two intranasal devices are joined together by a band that bows forward of the flange.

12. An intranasal device, comprising two arms, each arm comprising an anterior section shaped to fit snugly within a human nasal vestibule and a posterior section shaped to splint a septum corresponding to the human nasal vestibule, wherein the anterior sections of the two arms are joined together.

13. The intranasal device of claim 12, wherein each arm has the anterior section and the posterior section joined by a flexible connector.

14. The intranasal device of claim 12, wherein the anterior sections of the two arms have distinct, mirror image shapes that correspond respectively to anatomies of left and right human nasal vestibules.

15. The intranasal device of claim 12, wherein for each arm the posterior section includes a platelike member that is integral with a breathing passage.

16. The intranasal device of claim 15, wherein one side of the breathing passage is provided by the platelike member and an opposite side of the breathing passage is provided by a semi tube.

17. A method of post-surgical treatment, the method comprising:
inserting an intranasal device into a patient's nostrils, wherein the intranasal device has two arms each comprising a nostril dilator and a nasal splint; and
maintaining the intranasal device in the patient's nostrils using a mechanical interface that does not pierce flesh;
wherein the two arms are joined by a first connector; and
the nostril dilators and the nasal splints are joined by second connectors, which are flexible.

18. The method of claim 17, further comprising limiting intrusion of the two arms into the patient's nostrils using flanges attached to the nostril dilators.

19. The method of claim 17, further comprising removing the intranasal device by pulling on the first connector, wherein the first connector is bowed forward of the columella.

20. The method of claim 17, further comprising removing the intranasal device by a method that includes inserting an object between the first connector and the patient's columella and pulling on the intranasal device via the object.
